(19) United States
(12) Patent Application Publication
(10) Pub. No.: US 2005/0149272 A1
(43) Pub. Date: Jul. 7, 2005

(54) Method for sequencing polynucleotides

(76) Inventors: Itshack Pe' Er, Cambridge, MA (US); Ron Shamir, Rehovot (IL); Naama Arbili, Givatayim (IL)

Correspondence Address: NATH & ASSOCIATES, PLLC, Sixth Floor, 1030 15th Street, N.W., Washington, DC, 20005, US

(21) Appl. No.: 10/937,740

(22) Filed: Sep. 10, 2004

Related U.S. Application Data
(60) Provisional application No. 60/501,579, filed on Sep. 10, 2003.

(51) Int. Cl. C12Q 1/68; G06F 19/00; G01N 33/48; G01N 33/50

(52) U.S. Cl. 702/20

(57) ABSTRACT

A method for obtaining a candidate nucleotide sequence S indicative of a sequence of a target polynucleotide molecule that produces a hybridization signal I(\vec{x}) upon incubation with a polynucleotide \vec{x} for each polynucleotide \vec{x} in a set E of polynucleotides. For each polynucleotide \vec{x} in the set E of polynucleotides, a probability P₀(\vec{x}) of the hybridization signal I(\vec{x}) when the sequence \vec{x} is not complementary to a subsequence of T and a probability P₁(\vec{x}) of the hybridization signal when the sequence \vec{x} is complementary to a subsequence of T are obtained; so as to obtain a probabilistic spectrum (PS) of T. A score is then assigned to each of a plurality of candidate nucleotide sequences that is being based upon the probabilistic spectrum and upon a reference nucleotide sequence H. A candidate nucleotide sequence having an essentially maximal score is selected and one or more low confidence intervals and one or more reliable intervals in the selected candidate nucleotide sequence are identified. For each low confidence interval detected in the selected candidate nucleotide sequence, a score is assigned to each of a plurality of candidate nucleotide sequences of the low confidence region, where the score is based upon a probabilistic spectrum obtained by filtering from the PS signals the signals present in the reliable regions; and upon an interval of the reference nucleotide sequence H homologous with the low confidence interval. A candidate nucleotide sequence having an essentially maximal score is then selected. A revised candidate sequence S′ is then obtained indicative of the sequence of the target polynucleotide molecule T by substituting the sequence of the low confidence region in the candidate sequence S with the selected candidate sequence.

This application claims the benefit of prior U.S. provisional patent application No. 60/501,579 filed Sep. 10, 2003, the contents of which are hereby incorporated by reference in their entirety.

### FIELD OF THE INVENTION

This invention relates to computational methods in molecular biology, and more specifically to methods for determining the sequence of a polynucleotide.

### BACKGROUND OF THE INVENTION

Sequencing by hybridization (SBH) is a method for sequencing a polynucleotide such as a DNA molecule (Bains & Smith 1988, Lysov et al. 1988, Southern 1988, Drmanac and Crkvenjakov 1987, Macevics 1989). In this method, a chip, or microarray, is used consisting of a surface upon which all possible oligonucleotide probes of a particular length k (referred to herein as “k-mers”) are immobilized (Southern 1996). The DNA molecule whose sequence is to be determined, referred to as the “target molecule”, is allowed to hybridize to the k-mers on the chip. The target molecule and the k-mers on the chip may all be single stranded molecules. Alternatively, a double stranded target may first be cut into fragments having single stranded “sticky ends”, and the k-mers on the chip may be the sticky ends of double stranded molecules. Ideally, a single stranded target or the sticky end of a double stranded target hybridizes to a k-mer on the chip if and only if the sequence complementary to the k-mer occurs somewhere in the target sequence or the sticky end. Thus, in principle, it is possible to experimentally determine the “k-spectrum” of the target (the set of all k-long substrings present in the target). In practice, however, the data are ambiguous due to the ability of the target to bind to k-mers that are only partially complementary to one of its substrings. Thus, any binarization of the hybridization signal will contain errors.

The goal of SBH is to determine the target sequence from the target spectrum. However, even if the target spectrum were error free, the target sequence is not uniquely determined by the spectrum. If the number of sequences consistent with the spectrum is large, there is no satisfactory method to select the true sequence. Theoretical analysis and simulations (Southern et al., 1992, Pevzner and Lipshutz 1994) have shown that even when the spectrum is errorless and the correct multiplicity of each k-mer in the target sequence is known, the average length of a uniquely reconstructible target sequence using a chip of 8-mers is only about two hundred nucleotides, far below the length of a DNA molecule that may be sequenced by electrophoresis.

Let Σ=(A,C,G,T) designate the set of nucleotides composing a DNA molecule. M=4 is the “alphabet size”. A DNA sequence is a string over Σ which is denoted herein between braces (< >). The k-spectrum of a target sequence T of length L, T=<t₁, t₂, . . . t_{L}>, is the set of all k-long substrings (k-mers) of T. For each k-mer \vec{x}=<X₁, x₂, . . . Xₖ> in Σ^{k}, we define T (\vec{x})to be 1 if \vec{x} is a substring of T, and 0 otherwise. We denote K=M^{k}, the number of k-mers. A hybridization experiment measures, for each k-mer \vec{x} in Σ^{k}, an intensity of its hybridization with the target.

The result of an SBH experiment may be described by a graph in which each candidate target sequence is represented as a path in a graph (Pevzner et al., 1989). The graph is a directed de-Bruijn graph G(V,E) whose vertices are labeled by all the (k-l)-mers (the set of vertices V=Σ^{k-1}), and its edges are labeled by k-mers, (the set of edges E=Σ^{k}). The edge labeled <x₁, x₂ . . . xₖ> connects the vertex <x₁, x₂ . . . xₖ₋₁> to the vertex <x₂ . . . xₖ>. There is a 1-1 correspondence between L-long candidate target sequences and (L−k+1)-long paths in G, whose edge labels comprise the target spectrum. Hereafter, we interchangeably refer to edges and their labels, and also to sequences and their corresponding paths.

Since k-mers may reoccur in the target sequence, the paths do not have to be simple. When the spectrum is perfect and the multiplicities of the k-mers in the spectrum are known, every solution is an Eulerian path (Pevzner et al. 1989). In practice, however, the spectrum is not perfect and the multplicities are not known.

Alternative chip designs have been suggested, often assuming additional information, in order to reduce the ambiguity of the hybridization-based reconstruction.

SBH is limited by ambiguity in target reconstruction. Depending on k and on the target length, there may be several—or many—sequences, having the same spectrum and are thus indistinguishable by SBH. Hence, spectrum data do not contain sufficient information to unambiguously sequence targets of reasonable lengths (Pevzner, 1989; Pevzner and Lipshutz, 1994). Alternative sources of information have been suggested to complement the spectrum data.

One possible source of complementary information for SBH is a reference sequence. Genomic sequence data are now abundant. The genomes of more than a hundred species including human have already been sequenced. Despite this profusion of data, sequencing is still a routine task in laboratory work. This demand for sequencing is to a large extent targeted at molecules whose nucleic acid sequences are approximately known in advance. This is the case in validation of sequences, in cDNA sequencing, and in the detection and typing of polymorphisms or germline/somatic mutations. All these tasks can be categorized as “re-sequencing” tasks, i.e., the determination of a nucleotide sequence which is known to be a variant of some previously sequenced reference molecule. This promotes re-sequencing as a key endeavor in today's biology.

Nucleotide sequences from different sources may resemble each other, due to a common ancestral gene. This phenomenon is encountered within a species, between duplicated regions within a genome, and between individuals within a population. Small differences in sequences, referred to as “Single Nucleotide Polymorphisms” or SNPs, efficiently serve as genetic markers that are useful in medicine. Thus the detection and genotyping of SNPs has become an important task of human geneticists. The evolution of homologous sequences from a common ancestral gene is mainly due to nucleotide substitution. Insertions and deletions of nucleotides are also known to have occurred during evolution of homologous sequences, though at lower rates.

The identification of millions of human genetic polymorphisms and the mapping of all common human haplotypes has led to a situation in which all common sequence variations have been mapped. Nevertheless, due to the more modern expansion of the human race, much of the observed variation is comprised of rare polymorphisms and familial mutations. To determine the correct alleles of a certain locus borne by a specific individual it is thus insufficient to type only known single nucleotide polymorphisms (SNPs) that are abundant in the population: one would ultimately need to detect sporadic variations as well, and so, for many studies, complete re-sequencing will remain a key task in accurate genetic typing of individuals.

Resequencing by Hybridization (RSBH) is used to refer to reconstructing a target sequence using its spectrum and a known reference sequence that is presumed to be similar to the target sequence (Pe'er and Shamir, 2000, Pe'er et al., 2002). U.S. application Ser. No. 09/643,407, incorporated herein in its entirety by reference, discloses a computational method, referred to as “Spectrum Alignment” in which experimental spectrum data obtained from a DNA chip are combined with sequence information of a reference DNA molecule. A probabilistic representation of the reference sequence information and the spectrum signals is used to compute the most likely target sequence given these data. The reference molecule is preferably a molecule believed to be homologous with the target. For example, the target sequence may be a mutant gene and the reference sequence the previously sequenced normal gene. As another example, the target sequence may be a human gene and the reference sequence the homologous gene in another organism. A score is defined for each sequence in a set of candidate target sequences based upon a simultaneous comparison of the candidate sequence with the spectrum and with the reference sequence. A candidate target sequence is then selected having a essentially maximal score. Calculating the score does not require knowledge of the multiplicities of the k-mers in the k-spectrum. Moreover, the score does not assume that the spectrum is perfect.

In spectrum alignment, the hybridization of the target T with a k-mer on the DNA chip complementary to \vec{x} is described by probabilities P₀(\vec{x}) and P₁(\vec{x}) of the observed hybridization signal when T(\vec{x})=0, and T(\vec{x})=1, respectively. The results of a hybridization experiment are described by the “probabilistic spectrum” (PS ) defined as the pair (P₀,P₁) of functions Pᵢ: Σ^{k}→[0, 1]. If the experiment were perfect, i.e., if P₀ (\vec{x}) and P₁(\vec{x}) are either 0 or 1 with P₀(\vec{x})+P₁(\vec{x})≡-1, then the PS would represent the k-spectrum. In practice, however, P₀(\vec{x}) and P₁(\vec{x}) are both positive. There is thus a chance 1−P₀(\vec{x}) for a false positive (a k-mer (\vec{x}) not occurring in T, whose complementary sequence produces a hybridization signal indicative of hybridization) and a chance 1−P₁(\vec{x}) for a false negative (a k-mer (\vec{x}) occurring in T, whose complementary sequence produces a signal indicative of no hybridization).

The probability of obtaining a specific spectrum PS when T is used as the target is referred to as the “experimental likelihood”. The experimental likelihood is calculated assuming that the hybridization results of the target to different k-mer probes are mutually independent. For example, an experimental likelihood Le(t) may be used that does not assume knowledge of the multiplicities of each k-mer in the sequence. L^{e}(\hat{T}) is given by:

$${L}^{e} ( \hat{T} ) = Prob ( PS \vert \hat{T} ) = {\prod }_{\vec{s} ∈ {\sum }^{k}} {P}_{\hat{T} ( \vec{x} )} ( \vec{x} ) \qquad ( 1 )$$

Taking logarithms and defining

$$ω ( \vec{x} ) = log \frac{{P}_{1} ( \vec{x} )}{{P}_{0} ( \vec{x} )}$$

we can write:

$$\begin{matrix}log {P}_{\hat{T} ( \vec{x} )} ( \vec{x} ) = \{ \begin{matrix}log {P}_{0} ( \vec{x} ) & if \hat{T} ( \vec{x} ) = 0 \\ log {P}_{0} ( \vec{x} ) + ω ( \vec{x} ) & if \hat{T} ( \vec{x} ) = 1.\end{matrix} & ( 2 a ) \\ Hence , & \\ log {L}^{e} ( \hat{T} ) = {\sum }_{\vec{x} ∈ {\sum }^{k}} log {P}_{0} ( \vec{x} ) + {\sum }_{\hat{T} ( \vec{x} ) = 1} ω ( \vec{x} ) . & ( 2 b )\end{matrix}$$

The first term is a constant (independent of \hat{T}), and is omitted hereafter.

As another example, an approximate likelihood \tilde{L}(\hat{T}) may be used, that is defined as follows: Let p=e₀, . . . , e_{L-k} be the path in G corresponding to \hat{T} and define

$$log {\tilde{L}}^{e} ( \hat{T} ) = {\sum }_{i = 0}^{L - k} ω ( {e}_{i} ) . \qquad ( 3 )$$

\tilde{L}^{e}(\hat{T})=L^{e}(\hat{T}) for a path in which all edges have a multiplicity of 1, and is otherwise an approximation to L^{e}(\hat{T}). \tilde{L}^{e}(\hat{T}) has the advantage of being easily computable in a recursive manner:

$$log {\tilde{L}}^{e} ( {e}_{0} , … {e}_{l} ) = log {\tilde{L}}^{e} ( {e}_{0} , … {e}_{l - 1} ) + ω ( {e}_{l} ) \qquad ( 4 )$$

As yet another example, an experimental likelihood L^{e}(\hat{T}) may be used that takes into account the multiplicities of edges. In this case, the probabilistic spectrum consists of probabilities Pᵢ(\vec{x}), denoting the probability of the observed hybridization signal when the multiplicity of \vec{x} in the target is i. L^{e}(\hat{T}) is defined by:

$$\underline{{L}^{e}} ( \hat{T} ) = Prob ( PS \vert \hat{T} ) = {\prod }_{\vec{x} ∈ {\sum }^{k}} {P}_{\underline{\hat{T}} ( \vec{x} )} ( \vec{x} ) \qquad ( 4 b )$$

where \hat{T}(\vec{x}) is an indicator of whether \vec{x} occurs in \hat{T}.

When the target T=<t₁ . . . t₁> is a mutant sequence whose wild type sequence H=<h₁ . . . h₁> has already been sequenced, the wild type sequence H may be used as a reference molecule in spectrum alignment. In this case, the H and T usually differ from each other by nucleotide substitutions without insertions or deletions (indels). This would be the case, for instance, when one expects that nucleotide substitutions are the only cause of variability between H and T (statistically, substitutions are much more prevalent than indels). A set of M×M position specific substitution matrices M⁽¹⁾, . . . , A⁽¹⁾ are used, where for each position j along the sequence:
M^{(j)}[i,i′]=Prob(tⱼ=i|hⱼ=i′) (5)
for nucleotides i and i′∈Σ.

The matrices M^{(j)} may be the same for all j, or may different for different positions j. The matrices M^{(j)} are used to calculate a distribution on the space of possible target sequences. This “prior distribution for ungapped homology”, D^{u}, is given, for each candidate target sequence T by:

$${D}^{u} ( \hat{T} ) = Prob ( \hat{T} \vert H ) = {\prod }_{j = 1}^{l} {M}^{( j )} [ {t}_{j} , {h}_{j} ] \qquad ( 6 )$$

One may recursively compute:
D^{u}(<t₁ . . . tⱼ>)=(<t₁ . . . tⱼ₋₁>)·M^{(j)}[tⱼ,hⱼ] (7)
We denote L^{(j)}[x, y]≡log M^{(j)}[x, y].

The probability of a candidate target sequence \hat{T}, given the probability spectrum PS and the reference sequence H is:

$$Prob ( \hat{T} \vert H , PS ) = \frac{Prob ( H ) \cdot Prob ( \hat{T} \vert H ) \cdot Prob ( PS \vert H , \hat{T} )}{Prob ( H , PS )} \qquad ( 8 )$$

Given \hat{T}, the hybridization signal is independent of H:
P rob(PS|H,\hat{T})=P rob(PS|\hat{T})

Thus, omitting the constant

$$\frac{Prob ( H )}{Prob ( H , PS )}$$

we can write:
P rob(\hat{T}|H,PS)≅D^{u}(\hat{T})·L^{e}(\hat{T}) (9a)
P rob(\hat{T}|H,PS)≅D^{u}(\hat{T})·\tilde{L}^{e}(\hat{T}) (9b)
or
P rob(\hat{T}|H,PS)≅D^{u}(\hat{T})·L^{e}(\hat{T}) (9c)

Taking logarithms, the following “ungapped scores” of a candidate target are obtained:
S core₁^{u}(\hat{T})=log L^{e}(\hat{T})+log D^{u}(\hat{T}) (10a)
S core₂^{u}(\hat{T})=log \tilde{L}^{e}(\hat{T})+log D^{u}(\hat{T}) (10B)
S core₃^{u}(\hat{T})=log L^{e}(\hat{T})+log D^{u}(\hat{T}) (10c)

With Score^{u}₁,Score^{u}₂ or Score^{u}₃, the higher the score of a sequence \hat{T}, the more likely it is to be the target sequence. Methods for finding the highest scoring candidate sequence are disclosed in U.S. application Ser. No. 09/643,407. (When handling probabilities, some of which are perfect, problems of division by zero might occur. This is avoided by implicitly perturbing probabilities 0 and 1 to ε and 1-ε.)

The term “resequencing” implies that one has significant information on the reference, thus determination of the target sequence should avoid complete sequence determination de novo. One strategy for re-sequencing is by use of arrayed short probes. An array containing all possible probe sequences of a particular length can serve as a universal assay for all possible target sequences. In order to be economical, one should minimize probe number, and therefore probe length. However, shorter probes can reduce accuracy of the assay, so robust assay conditions and analytical processes need to be developed in concert with this simplified array approach.

### SUMMARY OF THE INVENTION

In the following description and set of claims, two parameters are considered to be equivalent to each other if they are proportional to each other.

In the following description, the invention is described in relation to the sequencing of polynucleotides. This is by way of example only, and the invention may be used in any polymer sequencing application such as the sequencing of polypeptides.

The present invention provides a method of resequencing in which spectrum alignment is applied iteratively. In accordance with the invention, after each resequencing step, putative incorrect regions in the sequence are identified having a likelihood below a predetermined threshold. The putative incorrect regions are referred to herein as “low confidence intervals”. Each iteration step re-sequences the sequence of the focus regions identified in the sequence produced by the previous iteration step, assuming correctness of the rest of the reconstructed sequence. This is done in order to correctly interpret probe signals that are positives, but are due to a match that occurred outside the focus region.

As stated above, the likelihood score Score^{u}(T) of a target sequence T only approximates the true likelihood score. To allow efficient computation, it adds the weight of each edge (subsequence corresponding to a probe) along the putative target sequence as many times as it appears along that sequence, whereas a mathematically precise (albeit computationally expensive) computation would add each such weight only once. The likelihood score therefore deviates from the exact likelihood score whenever an edge is revisited along the sequence. The shorter the target sequence, the rarer this deviation event is. Thus, in accordance with the invention, the sequence produced by the previous iteration step is divided into “reliable intervals”, and “low confidence intervals”. The reliable intervals are those intervals of the sequence whose average per-edge contribution to the likelihood ratio is over a predetermined threshold t. The reliable intervals are presumed to be accurately sequenced. The low confidence intervals are those intervals of the sequence whose average per-edge contribution to the likelihood ratio is not over the predetermined threshold t. The low confidence intervals are presumed to be incorrectly sequenced and which are to be resequenced in the subsequent interation. The union of all of the low confidence intervals of the sequence is referred to herein as “the focus region”. Assuming the correctness of the sequence in the reliable intervals implies which edges appear in the reliable intervals. This allows the deviation of the likelihood score from the true likelihood it approximates to be calculated. This process is referred to herein as “filtering the spectrum”.

As stated above, the output of spectrum alignment is a path in the de-Bruijn graph, i.e. a series of edges, along which the likelihood score is maximized. In one embodiment, low confidence intervals are found by exhaustively checking, for each interval of the sequence whether its score exceeds t.

The spectrum is a set of weights assigned to edges of the de-Bruijn graph. Given a spectrum of the target generated by the previous iteration step, and partition of the target into reliable and low confidence intervals, the spectrum is transformed to account for probes (edges) that are known to be part of the sequence in reliable regions, and the computation does not add their weight again to the score of other regions, as this weight is already part of the score of a reliable region. This is done by setting the weight of each of those probes to zero, so as not to consider their already-added weight again.

For each low confidence interval flanked by two reliable intervals found in the target sequence of the previous iteration steps, the homologous interval of the reference sequence corresponding to the low confidence interval is then determined from the homology of the target sequence of the previous iteration step and the reference sequence. The low confidence interval is then resequenced by spectrum alignment using this homologous interval of the reference sequence as the reference sequence of the resequencing together with the filtered spectrum of the low confidence interval. The starting and ending probes for the spectrum alignment are implied by the flanking, reliable regions at both ends of the low confidence interval.

At each iteration step, all of the identified low confidence intervals are resequenced, as described above, so as to resequence the entire focus region. The iteration is preferably repeated a number of times until no low confidence intervals are found in the sequence.

Formally, we denote the basic Spectrum Alignment is treated as a procedure, called SA, that obtains a target sequence Tₖ₊₁ . . . T₁₋ₖ and a cumulative likelihood function L of sub-sequences thereof. The inputs to SA inputs are the spectrum, S, the flanking sequences T₁ . . . Tₖ, and T₁₋ₖ₊₁ . . . T₁, and the homologous sequence Hₖ₊₁, . . . H₁₋ₖ. The enhanced procedure is therefore as follows:
1. Set A⁰₁←k+1, B⁰₁←1-1, N⁰←1, S⁰=S, i←0
_{2.} For each j=1 . . . Nⁱ
   a. Set a=Aⁱⱼb=Bⁱⱼ
   b. Compute (Tₐ . . . T_{b}, L)=SA(Sⁱ, Tₐ₋ₖ . . . Tₐ₋₁,T_{b+1} . . . T_{b+k},Hₖ₊₁ . . . H₁₋ₖ)
3. Set i ←i+1
_{4.} Find a set low confidence intervals along T using the likelihood function L. Let n=Nⁱ be the number of such intervals, and let Aⁱ₁, . . . Aⁱₙ and Bⁱ₁ . . . Bⁱₙ denote their starting/ending points, respectively. If there are no such regions—halt.
5. Compute Sⁱ, the filtered spectrum, by setting all Sⁱ⁻¹ spectrum entries corresponding to high confidence intervals, to zero.
6. Goto step 2.

Successful re-sequencing of 100 bp fragments using pentanucleotide probes was obtained as disclosed in Pe'er et al., 2003 and U.S. patent Ser. No. 09/643,407. This suits several key applications for re-sequencing, in which the sequence of a target exon, for example, may differ from its reference at many polymorphic or mutable sites. Such applications include genetic, diagnostic tests for highly polymorphic genes like CFRR that has over a thousand known mutations, many of them treatable upon proper diagnosis. An additional application involves detecting somatic mutations in onco-related genes. Accurate typing of pathogens can be also be achieved, by re-sequencing genes that are common to all candidate pathogens (e.g., 16S RNA).

The invention may be carried out using spectrum data obtained via any of several technologies. For example, a ligation assay (Gunderson et al., 1998) may be used, where a very detailed spectrum of relatively long oligonucleotides is obtained, at the price of having to pool several probes to one measured signal.

Use of 5-mer probes in the method of the invention accurately sequences polynucleotides op to 100 bp in length. In order to sequence fragments longer by an order of magnitude, the probe length may be scaled to include all-8-mers, or even all-9-mers; arrays that are feasible with some current industrial technologies. Indeed, simulation studies (Pe'er et al., 2002) indicate that the feasible target length for re-sequencing approximately doubles when increasing by one the probe length in a universal array, even without taking into account any potential increase in accuracy due to longer probes.

Longer probes may be used together with more stringent hybridization/extension conditions in order to reduce spurious biochemical outcomes. More intense, and more sensitive detection molecules and scanning technologies may be used to improve detection of weaker signals, and increase the sensitivity well beyond the simple method of incorporation of singly labeled fluorescent nucleotides. Any of these alternatives could be used in the present invention in order to increase accuracy, and enhance the overall fidelity of the re-sequencing process.

The invention may be used with the 5-mer resequencing technique to explore a small number of differences, which is the goal in some resequencing studies. In this case, detection of small variations with respect to the reference sequence becomes far more important.

This is achieved by examining potential improvements to the overall likelihood score by putatively assuming heterozygocity at each polymorphic sequence position. The possibly improved likelihood score is rapidly computed using the filtered spectrum introduced above. In this application, a pair of sequences is sought, corresponding to a pair of paths in the Spectrum Alignment graph, that maximize the likelihood of the signals under the assumption of the two corresponding haplotypes. This likelihood is an expression which sums up individual edge contributions, very similarly to the standard homozygous score. In practice, the two haplotypes are expected to be quite similar to each other. Therefore the two corresponding paths are intertwined, and often overlap in many edges. The resolution of one haplotype can be performed as in the homozygous case. Regions where the two paths are distinct in a segment are resolved by segment fashion. When examining such a segment, one can look for potential heterozygocity by using the distilled spectrum machinery to filter out the spectrum of the first haplotype (Pe'er et al., 2003).

In another of its aspects, the invention also provides a method for determining the distributions P₀ and P₁. In one embodiment of this aspect of the invention, referred to herein as “Per-probe Training”, P₁(x) is evaluated as follows. When there are sufficient examples of fluorescent signals for the probe x with known positive match to some known target, the mean signal μ₁(x) for the matched probe is evaluated, and its standard deviation is σ₁(x) determined. A goodness-of-fit test does not reject the hypothesis that samples are normally distributed. P₁(x) is then set to the p-value for signal s(x) to be drawn from a normal distribution with mean μ₁(x) and standard deviation σ₁(x). Evaluation of P₀(x) is done similarly. For convenience, it may be assumed that σ(x)=σ₁(x)=σ₀(x) and then σ(x) is evaluated on the two sets of samples.

When sufficiently many samples of positive/negative matches to the probe x are not available, the occurrence count of positive/negative matches to the probe x is enriched by adding to it the count of another probe y, whose signals are similarly distributed, but whose counts are not sparse. For each candidate probe y we use its computed normal distributions, N(μ₁(y),σ²(y)) and N(μ₁(y),σ²(y)), to evaluate the likelihood of the observed matched and unmatched, respectively, signals for x. The probe y=y* that maximizes this likelihood is chosen and its counts are added to those of x. The combined count is used to evaluate expectancies μ₁, μ₀ and the standard deviation σ for x that together define the normal distributions of its matched/unmatched signals.

In another embodiment of this aspect of the invention, referred to herein as “Probe-Independent Training”, the distributions P₀ and P₁ are learned in an unsupervised manner, an alternative strategy is employed, which does not build on experience with previous assays, and does not fit the distribution for each probe. Instead, the distributions of signals with positively and negatively matched probes in the current dataset are utilized.

Obviously, it is not possible to know in advance for the current dataset whether a probe is perfectly matched by the target or not, as the target is yet unknown. However, the probability of that event with respect to a random target that is similar to the reference sequence can be evaluated since the true target is presumed to be similar to the reference sequence. Such random targets can be drawn using a hidden Markov Model (HMM, Durbin et al. 1998), which models the probabilistic space of such sequences. We can generate a large number of candidate targets, and average the matched/unmatched status of the probe \vec{x} as follows: the probability of a perfect match is empirically estimated based on all randomized targets, as the fraction of probes attaining a certain signal among perfectly matched probes:

$${P}_{1} ( x ) = \frac{{\sum }_{random target t} ( {N}^{<} ( t , s ( x ) ) + 0.5 {N}^{=} ( t , s ( x ) ) )}{{\sum }_{random target t} {N}^{<} ( t , ∞ )} \\ {P}_{1} ( x ) = \frac{0.5 + \# Experiments with perfect match for x and signal < s ( x )}{1 + \# Experiments with perfect match for x} \qquad ( Eq . 1 )$$

P₀(x) is estimated analogously,
where N²¹ (t,s) denotes the number of experiments perfectly matching x displaying a signal below s, and N⁼(t,s) denotes the number of experiments perfectly matching \vec{x} displaying equal to s.

The invention may also be used to detect heterozygotes by the same iterative principle used to improve performance in potentially incorrect regions. Potentially heterozygous positions may be identified by a local decrease in the likelihood difference between the most likely sequence and the second most likely (both of which may be identified by Spectrum Alignment and its variants). In such situations iteratively applying Spectrum Alignment locally is used, for re-sequencing a second allele.

The invention may also be used to analyze data from technologies of pooled probes. In such technologies the experimental information per pool is a signal essentially representing the maximal signal of all probes in the pool. For each pool X, yielding a signal intensity s(X), we can thus write P₁(X)=prob(signal is s(X)| any of the probes in X matches the target)≈max_{x∈X}P₁(x). Similarly, P₀(X)≈min_{x∈X}P₀(x). While Pᵢ(X) is available from the data and the signal distribution, Pᵢ(x) is the quantity required for Spectrum Alignment analysis. The former quantity can be substituted for the latter during analysis. While this is an approximation, the iterative algorithm of the invention gradually improves its accuracy by focusing at specific regions and accounting for all probe signals for matches outside that region. Since most pools consist of no more than one matched probe, when this match is accounted for, Pᵢ(\vec{x})≈Pᵢ(X) for all x∈X.

It will also be understood that the system according to the invention may be a suitably programmed computer. Likewise, the invention contemplates a computer program being readable by a computer for executing the method of the invention. The invention further contemplates a machine-readable memory tangibly embodying a program of instructions executable by the machine for executing the method of the invention.

Thus, in its first aspect the invention provides a method for obtaining a candidate nucleotide sequence S, the candidate nucleotide sequence S being indicative of a sequence of a target polynucleotide molecule T, T producing a hybridization signal I(\vec{x}) upon incubating T with a polynucleotide \vec{x} for each polynucleotide \vec{x} in a set E of polynucleotides, the method comprising the steps of:
(a) for each polynucleotide \vec{x} in the set E of polynucleotides, obtaining a probability P₀(\vec{x}) of the hybridization signal I(\vec{x}) when the sequence \vec{x} is not complementary to a subsequence of T and a probability Pₗ(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
   (ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;
   (eb) selecting one or more candidate nucleotide sequences having an essentially maximal score; and
   (ec) determining a revised candidate sequence S′ indicative of the sequence of the target polynucleotide molecule T by substituting the sequence of the low confidence region in the candidate sequence S with the candidate sequence selected in step (eb).

In its second aspect, the invention provides a program storage device readable by machine, tangibly embodying a program of instruction executable by the machine to perform method steps for obtaining a candidate nucleotide sequence S, the candidate nucleotide sequence S being indicative of a sequence of a target polynucleotide molecule T, T producing a hybridization signal I(\vec{x}) upon incubating T with a polynucleotide \vec{x} for each polynucleotide \vec{x} in a set E of polynucleotides, the method comprising the steps of:
(a) for each polynucleotide \vec{x} in the set E of polynucleotides, obtaining a probability P₀(\vec{x}) of the hybridization signal I(\vec{x}) when the sequence \vec{x} is not complementary to a subsequence of T and a probability Pₗ(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
(ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;
(eb) selecting one or more candidate nucleotide sequences having an essentially maximal score; and
(ec) determining a revised candidate sequence S′ indicative of the sequence of the target polynucleotide molecule T by substituting the sequence of the low confidence region in the candidate sequence S with the candidate sequence selected in step (eb).

In its third aspect, the invention provides a computer program product comprising a computer useable medium having computer readable program code embodied therein for obtaining a candidate nucleotide sequence S, the candidate nucleotide sequence S being indicative of a sequence of a target polynucleotide molecule T, T producing a hybridization signal I(\vec{x}) upon incubating T with a polynucleotide \vec{x} for each polynucleotide \vec{x} in a set E of polynucleotides, the computer program product comprising:
(a) for each polynucleotide \vec{x} in the set E of polynucleotides, obtaining a probability P₀(\vec{x}) of the hybridization signal I(\vec{x}) when the sequence \vec{x} is not complementary to a subsequence of T and a probability Pₗ(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
   (ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;;
   (eb) selecting one or more candidate nucleotide sequences having an essentially maximal score; and
   (ec) determining a revised candidate sequence S′ indicative of the sequence of the target polynucleotide molecule T by substituting the sequence of the low confidence region in the candidate sequence S with the candidate sequence selected in step (eb).

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows a fluorescence confocal microscope scan of a reacted universal array (Dataset 6, experiment 6) having 992 different unique probes, 32 duplicated probes and 96 positive and negative control probes.

FIG. 2 shows signal level distributions for matched and unmatched probes using data collected from datasets 2-6, in which, for each level of the fluorescent signal, the black plot displays the fraction of matched probes that produced at least this level of signal, and the gray curve displays the fraction of unmatched probes that produced at most this level of signal.;

FIG. 3 shows signals of two specific probes using data collected from datasets 2-6: TTAGC, whose signals are extremely high, and CGTGA, whose signals are extremely low. For each level of the fluorescent signal, the number of matched (black bars) or unmatched (gray bars) probes that produced this level of signal is displayed. Every threshold rule for calling matched/unmatched by fluorescent signal level would either label all TTAGC probes as matched or all CGTGA probes unmatched. Nevertheless, analysis of each probe individually separates positive versus negative signals much better.

FIG. 4 shows re-sequencing performance using different training procedures. The training procedures are used for generating probe signal distributions in the Spectrum Alignment algorithms. Tests were performed on all the CF arrays (datasets 2-5). Bars represent success rate of genotype calls. For a genomic bi-allelic amplicon target, we count a polymorphism as successfully typed if both predicted alleles match those present in the sample. Half an error is reported for each allele mismatch. Mono-allelic synthetic targets (arrays 5-7 in datasets 2 and 3) were all successfully typed and counted as one success each. A. Probe-independent training based on the current experiment only (no prior data) B. Per-probe training, using the current dataset for probes with three or more matched and unmatched examples observed. For probes with fewer examples, an enrichment procedure is applied (see Methods). C. Per-probe training using all datasets. D. Per-probe training using all datasets except the dataset that contains the target.

FIG. 5 shows a summary of resequencing results for CFTR. The wildtype reference sequence is displayed along with callouts for statistics on the typing of sites with potential mutations found at specific nucleotides. In total, 60.5 out of 64 mutations were correctly typed in common SNP sites (white callouts). Two mutations were called in spurious sites (gray callouts).

FIG. 6 shows visualization of re-sequencing results by SNP-o-gram. A synthetic short target, with two known mutations(array 6, dataset 3) b A genomic target which is heterozygous for a single known mutation (array 2, dataset 4).

### DETAILED DESCRIPTION OF PREFERRED EMBODIMENTS

Materials and Methods

Target Molecules

Target samples included 10 synthetic double stranded DNA molecules of length 25-35 bp and 32 PCR amplicons of length 100-140 bp (see Tables 1 and 2).

TABLE 1
summary of datasets analyzed

| | | Experi- | | Total |
|---|---|---|---|---|
| | Number of | ments | | number of |
| Probe set | probes | per dataset | Datasets | experiments |
| Angiotensinogen | 176 unique | 6 | 1 | 6 |
| tiling | | | | |
| CFTR tiling | 176 | 7/8 | 2, 3, 4, 5 | 30 |
| | (166 unique) | | | |
| Universal | 1119 | 6 | 6 | 6 |
| | (1024 unique) | | | |

TABLE 2

| | Ex- | | | | | |
|---|---|---|---|---|---|---|
| | peri- | Target | | | | |
| Dataset | ment | Type¹ | Locus | From2 | To3 | Mutant3 |
| 1 | 1 | A | AGT4 | 4078 | 4177 | W |
| | 2 | A | AGT | 4078 | 4177 | ATG281ACG |
| | 3 | A | AGT | 4078 | 4177 | W |
| | 4 | A | AGT | 4078 | 4177 | ATG281ACG |
| | 5 | A | AGT | 4078 | 4177 | W |
| | 6 | A | AGT | 4078 | 4177 | ATG281ACG |
| 2 | 1 | A | CFTR5 | 107766 | 107863 | GGA542TGA |
| | 2 | A | CFTR | 107782 | 107891 | GGT551G[G/A]T |
| | 3 | A | CFTR | 107782 | 107891 | CGA553TGA |
| | 4 | A | CFTR | 107810 | 107917 | AGG560ACG |
| | 5 | S | CFTR | 107803 | 107827 | GGA542TGA |
| | 6 | S | CFTR | 107803 | 107827 | W |
| | 7 | S | CFTR | 107858 | 107881 | W |
| 3 | 1 | A | CFTR | 107766 | 107863 | GGA542TGA |
| | 2 | A | CFTR | 107782 | 107891 | GGT551G[G/A]T |
| | 3 | A | CFTR | 107782 | 107891 | CGA553TGA |
| | 4 | A | CFTR | 107810 | 107917 | AGG560ACG |
| | 5 | S | CFTR | 107834 | 107856 | W |
| | 6 | S | CFTR | 107834 | 107856 | GGT551GAT + |
| | | | | | | CGA553TGA |
| | 7 | S | CFTR | 107858 | 107881 | AGG560ACG |
| 4 | 1 | A | CFTR | 107782 | 107891 | GGA542TGA |
| | 2 | A | CFTR | 107782 | 107891 | GGT551G[G/A]T |
| | 3 | A | CFTR | 107825 | 107914 | CGA553TGA |
| | 4 | A | CFTR | 107825 | 107914 | AGG560ACG |
| | 5 | A | CFTR | 107795 | 107893 | CGA553TGA |
| | 6 | A | CFTR | 107766 | 107863 | W |
| | 7 | A | CFTR | 107810 | 107917 | GGT551G[G/A]T |
| | 8 | A | CFTR | 107766 | 107863 | GGA542TGA |
| 5 | 1 | A | CFTR | 107766 | 107863 | GGA542TGA |
| | 2 | A | CFTR | 107766 | 107863 | W |
| | 3 | A | CFTR | 107782 | 107891 | GGT551G[G/A]T |
| | 4 | A | CFTR | 107782 | 107891 | W |
| | 5 | A | CFTR | 107825 | 107914 | CGA553TGA |
| | 6 | A | CFTR | 107825 | 107914 | W |
| | 7 | A | CFTR | 107810 | 107917 | AGG560ACG |
| | 8 | A | CFTR | 107810 | 107917 | W |
| | 1 | S | Ch 186 | 44 | 78 | Base 19 A→G |
| | 2 | S | Ch 18 | 44 | 78 | W |
| | 3 | A | Ch 18 | 1 | 109 | Base 62 A→G |
| | 4 | A | Ch 18 | 1 | 109 | W |
| | 5 | S | CFTR | 107803 | 107827 | W |
| | 6 | S | CFTR | 107803 | 107827 | GGA542TGA |

²Offset (bp) from translation start site (coding sequences) or from segment start (non coding).
³Either the wildtype (W) or a mutant, which is denoted by the original codon, codon number and new codon (coding sequences) or bp number with base change (non coding). Samples that are heterozygous for a mutation are denoted by, e.g. [A/G].
⁴Genomic sequence at positions 769274 . . . 780916 of GI: 27477742.
⁵Genomic sequence at positions 42296576 . . . 42485274 of GI: 22050628.
⁶Genomic sequence at positions 136976 . . . 137084 of GI: 18677476.

PSA

The spectra of the targets in this embodiment were obtained using Polymerase Signaling Assay (PSA) (Liu et al., 2001; Head et a., 2001; Head et al., 2002). PSA uses a glass slide, onto which probes are spotted in an arrayed fashion. Plates were used having 192 spots each, where 16 spots are used as controls, and 176 spots each contain a unique 5-base probing sequence, representing 5-mers and sequence variations specifically related to the target sequence being tested. Used for analysis of AGT exon 2 and CFTR exon 11, these experiments simplify the approach from the true “universal array” of 5-mers. A complete universal array, which may be used for analysis of any arbitrary sequence, has a unique 5-base probe for each of the 4⁵=1024 possible pentanucleotide combinations. These larger arrays were constructed by using several sub-arrays. The probe-specific nucleotide combinations were designed to perfectly match every possible 5-mer segment along a target. Exact details of this assay are described for example in Liu et al., 2001; Head et a., 2001; Head et al., 2002, incorporated herein by reference.

Results

A series of blind tests were performed, in which the target sequence was unknown. One set of assays comprised simple genotyping tests, where the target sequence was either the wildtype or a single-nucleotide mutant thereof. Other assays were re-sequencing tests, wherein the target could have been any variant of the known reference sequence.

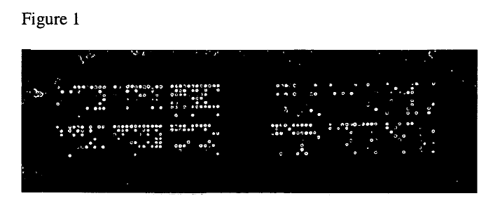

Partial, tiling arrays were constructed. Some of these arrays consisted of probes that tile variants of exon 2 of Angiotensinogen, while others tile exon 11 of CFTR. Universal arrays were also constructed and tested complete. Arrays were used arrays of 5-mer probes, for which only 1024 different oligonucleotides are needed. See Table 1 for the list of arrays used. Various target molecules were re-sequenced (see Table 2). To obtain as much specificity as possible from these short probes, the PSA protocol was applied (see Methods). The image, a confocal fluorescence scan, of one such universal array is presented in FIG. 1.

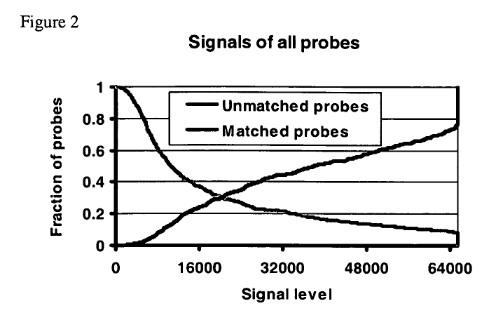

Arrayed PSA reactions produce datasets of raw fluorescent signals. When reconstructing a target sequence using Spectrum Alignment, the quantity of interest for each probe is the likelihood of a perfect match. More precisely, given the raw signal s(\vec{x}) for a probe x, one needs to compute the probabilities P₁(x)=Prob(s(x)|x is perfectly matched by the target) and P₀(x)=Prob(s(x)|x is not perfectly matched by the target). Although PSA provides cleaner signals than hybridization, the signals may still be very noisy. The observed noise might be due either to stochastic effects, causing variation in replicate observations of the same intensity, or to hidden variables that distinguish between signals. As shown below, both factors contribute to the signal distribution, and knowledge of some hidden variables, such as individual probe differences can be exploited, to improve signal analysis. Overall distributions of signals are presented in FIG. 2. These distributions, though obviously different, have a broad range of overlap. Consequently, a simple threshold value cannot effectively distinguish between matched probes and unmatched ones. Furthermore, even if we use the probabilities in FIG. 2, for most of the signal range, the matched and unmatched probabilities are of the same order of magnitude. Thus the log-likelihood term log[P₁(x)/P₀(x)] contributed by most probes is around zero, rendering the model statistically weak.

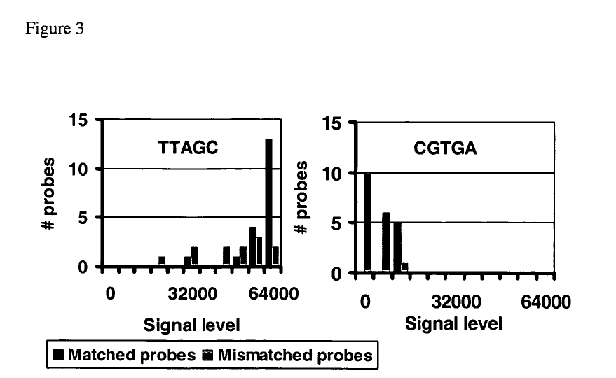

The weak separation of the P₀ and P₁ distributions can have two causes: Either the individual per-probe distributions are separated weakly for most probes, or they are separated, and their superposition causes the weak separation. Fortunately, as exemplified by FIG. 3, the latter case is in effect. For example, T-rich probes produce very high signals, due to the poly-A capture probes used in PSA (see Methods). Therefore, negative signals for such probes would be deemed positive according to the overall signal distribution, which is a mixture of many different per-probe distributions (see FIG. 2). This suggests empirically estimating P₀ and P₁ on a per probe basis. For each probe x, for each signal level s, we estimate the probability of observing a signal s(x) under the assumption of a perfect match in the target sequence. We assume such signals are normally distributed, with a probe-specific mean and variance, providing the distribution of P₁(x). The distribution of P₀(x) is analogously estimated.

Two scenarios were studied and tested. In one embodiment, each of the two distributions P₀ and P₁ is estimated by assuming that the two distributions are the same for all probes. This method is referred to herein as probe-independent training. In another embodiment that may be used in cases in which several arrays were assayed using the same protocol, but with different target molecules, individual signal distributions for each probe are estimated under an approximate assumption that these arrays are replicates of the same experiment. This embodiment method is referred to herein as per-probe training.

In probe-independent training, in the absence of any prior information on the signal distributions, the following approximation may be used. Many random targets are generated in simulation which are variants of the reference sequence, and statistics are collected on the signal distributions of matched and unmatched probes. In this manner, the statistical properties of the actual target sequence used in the assay is modeled, without having any further information about the actual biochemical outcome of known target variants. (See Methods).

In per-probe training, several arrays are used that were assayed using a similar reference, but with different mutations. This is the case, for example, for each individual dataset in Table 1, which used several arrays. This is also the case for all the datasets of the CFTR arrays that together constitute a much richer set. Thus, a number of experiments with extensive perfect match data are available. In order to resolve the target in a specific array, each probe is trained using all other arrays with match/mismatch for the current probe. The matched/unmatched signal levels are pooled for each probe from all arrays and obtain a richer distribution. When that distribution is not based on sufficiently many probe occurrences, that distribution may be enriched by that of another, similar probe (see Methods). As samples accumulate, richer and richer training sets can be built and exploited this way, so that statistical confidence of any single experiment increases.

The two training methods present a tradeoff: Probe-independent training uses a rich, yet coarse, set of observations, and forms a distribution that may be not representative of the specific probe. The per-probe method uses a finer set of observations, which may be too small a sample, and thus overfit the estimated distribution. We also consider a similar tradeoff with respect to the experiments used to learn the per-probe distribution: We compare results of analysis based on learning this distribution from the current dataset only, to learning based on all datasets, or on all other datasets except the current one.

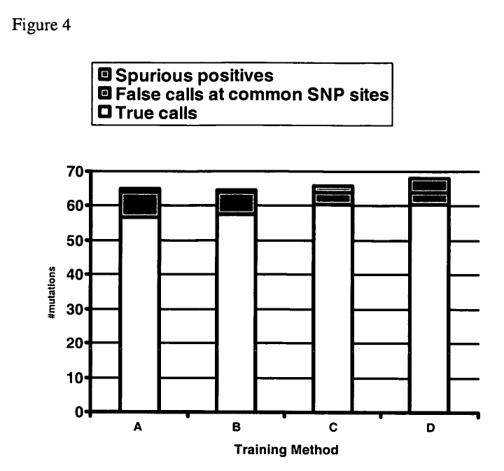

FIG. 4 presents a comparison of the results obtained by each of the training methods. The function log[P₁(x)/P₀(x)] was used as the per nucleotide scoring function. A threshold value of 3 was used to distinguish between low confidence intervals and reliable intervals. Per-probe analysis based on all other arrays is superior to probe-independent analysis based on the current dataset only. In per-probe methods, there is a tradeoff between training which is based only on the same dataset and training on all datasets: The more refined, but sparser training per dataset makes more false calls at known SNP sites, but reports less spurious false positives due to overfitting.

The estimated probabilities serve as input to the Spectrum Alignment computational engine. Table 3 presents results for blind tests of genotyping and for re-sequencing tests. For angiotensinogen exon 2, targets were either wildtype or mutated for a specific polymorphism. The algorithm was not calibrated beforehand with any prior information regarding the identity of this polymorphic site, i.e., the reference sequence model was considered to have an equal likelihood to contain a mutation at any point along the target sequence. The genotype call on this site was correct for 6 out of 6 samples, and no spurious calls were made (although permitted by the algorithm). Analysis for arrays in this dataset was carried out using probe-independent training. Although each of the 5-mer probes may not necessarily give an entirely specific assay signal, their joint analysis using the Spectrum Alignment algorithm (Pe'er et al., 2002) utilizes all the statistical information available to produce a strong, combined signal.

TABLE 3
genotyping results

| | Experi- | Re-sequencing | Correct | Log-likelihoods | |
|---|---|---|---|---|---|
| Dataset | ment | call⁹ | genotypes | Wildtype | Mutant |
| 1 | 1 | W | 1 | −205.847 | −221.579 |
| | 2 | ATG281ACG | 1 | −206.34 | −204.01 |
| | 3 | W | 1 | −206.37 | −220.155 |
| | 4 | ATG281ACG | 1 | −206.953 | −198.819 |
| | 5 | W | 1 | −205.631 | −220.109 |
| | 6 | ATG281ACG | 1 | −207.039 | −198.845 |
| 2 | 1 | GGA542TGA | 1 | −181.304 | −175.85 |
| | 2 | W | ½ | −204.646 | −199.807 |
| | 3 | CGA553TGA | 1 | −193.649 | −192.389 |
| | 4 | AGG560ACG | 1 | −213.685 | −210.591 |
| | 5 | GGA542TGA | 1 | −240.386 | −236.305 |
| | 6 | W | 1 | −216.133 | −232.219 |
| | 7 | W | 1 | −153.507 | −171.118 |
| 3 | 1 | GGA542TGA | 1 | −183.781 | −177.255 |
| | 2 | W | ½ | −219.014 | −218.487 |
| | 3 | CGA553TGA | 1 | −202.959 | −197.73 |
| | 4 | AGG560ACG | 1 | −208.909 | −200.895 |
| | 5 | W | 1 | −203.153 | −224.416 |
| | 6 | GGT551GAT + | 2 | −186.667 | −156.294 |
| | | CGA553TGA | | | |
| | 7 | AGG560ACG | 1 | −155.797 | −141.592 |
| 4 | 1 | W | 0 | −258.733 | −261.182 |
| | 2 | W | ½ | −198.028 | −195.828 |
| | 3 | CGA553TGA | 1 | −197.348 | −193.998 |
| | 4 | AGG560ACG | 1 | −203.601 | −200.474 |
| | 5 | CGA553TGA | 1 | −194.639 | −192.439 |
| | 6 | W | 1 | −178.632 | −191.412 |
| | 7 | W | ½ | −205.818 | −246.755 |
| | 8 | GGA542TGA | 1 | −243.99 | −238.935 |
| 5 | 1 | GGA542TGA | 1 | −248.925 | −239.479 |
| | 2 | W | 1 | −211.087 | −222.238 |
| | 3 | W | ½ | −246.786 | −255.151 |
| | 4 | W | 1 | −236.699 | −248.77 |
| | 5 | CGA553TGA | 1 | −212.363 | −209.091 |
| | 6 | W | 1 | −208.375 | −208.806 |
| | 7 | AGG560ACG | 1 | −221.538 | −220.552 |
| | 8 | CGA553CAA + | 0 | −258.038 | −255.874 |
| | | AGA555AGC | | | |
| 6 | 1 | Base 19 A→G | 1 | −1190.56 | −1181.98 |
| | 2 | W | 1 | −885.905 | −906.477 |
| | 3 | W | 0 | −907.967 | −912.53 |
| | 4 | W | 1 | −883.603 | −899.166 |
| | 5 | W | 1 | −766.15 | −781.939 |
| | 6 | GGA542TGA | 1 | −691.528 | −686.091 |

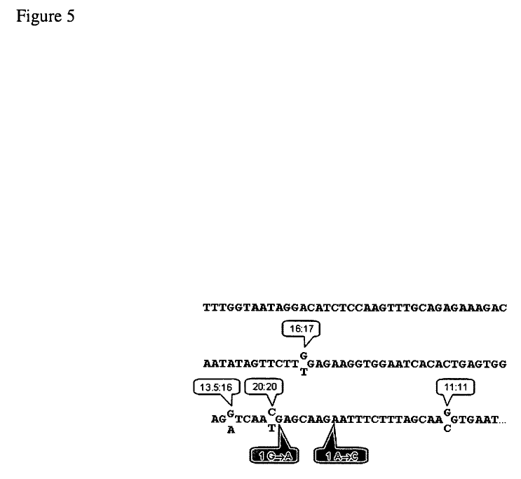

FIG. 5 presents results for the CFTR exon 11. For re-sequencing this target (with either partial or universal arrays), we used as reference not only the genomic sequence, but also known mutations from the Human Genome Mutation Database (www.hgmd.org). All together, in 30 arrays, 2.6 kb of DNA was re-sequenced. Out of 64 known polymorphisms, 60.5 (see FIG. 4) were correctly typed, and two additional spurious mutations were falsely detected. This true-positive rate of 95% is to be contrasted with the 30% error rate introduced by pentamer biochemistry (FIG. 2). Observe that this analysis was carried out without any attempt to detect heterozygocity. While genotyping does require the detection of heterozygotes (see Discussion). A first, simple approach to test the feasibility of our methodology was employed, which ignored heterozygocity, and therefore technically counted heterozygotes as errors. Out of the 56 homozygotes, only one error occurred.

A non-coding region on chromosome 18 was also re-sequenced by universal arrays (dataset 6, arrays 1-4). For this target sequence we had no prior knowledge of the mutant sites. For this segment we missed one of the mutations in four re-sequenced targets of total length of 300 bp. Both CFTR targets assayed with universal arrays (dataset 6, arrays 5 and 6) were successfully resequenced.

Although per-probe signal effects by per-probe training has been accounted for, the major source of remaining error appears to be systematic bias, rather than stochastic effects between replicates: most of the failed genotypes involve the GGT551G[G/A]T mutation. Thus, apparently, averaging many experiments will not be helpful in eliminating such errors, but further understanding and modeling of the causes of such systematic bias may solve the problem.

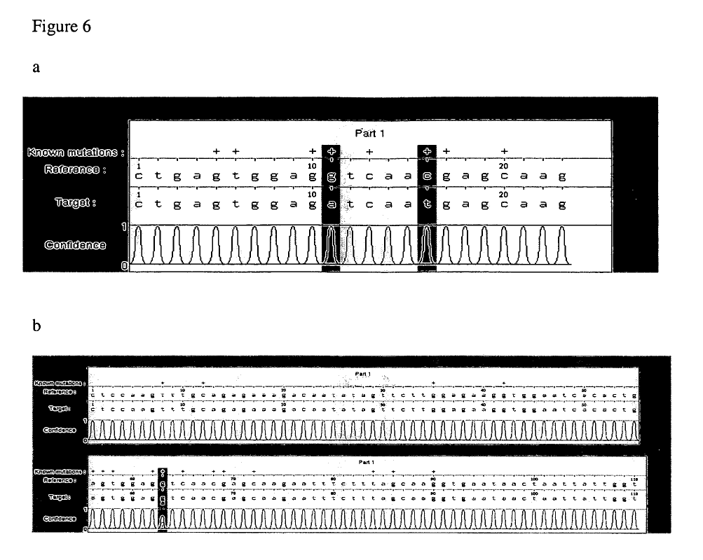

The Spectrum Alignment algorithm was implemented on both Windows and Unix platforms. The implementation incorporates a refined analysis of heterozygote samples, although the results presented were analyzed without this feature. The heterozygotes analysis would obviously need to be added for full functionality. In addition, a visualization tool was implemented, called SNP-o-gram, for presentation of re-sequencing results. This Windows application displays the reference and re-sequenced target, along with plots that indicate the likelihood of each basecall, similar to standard traces of gel-based sequencing machines. FIG. 6 displays the SNP-o-gram of two re-sequenced targets.

The following references are considered relevant to an understanding of the inventive subject matter, and their inclusion for such purpose is not an admission that such documents are material to patentability of the claimed subject matter, nor an admission that such documents are prior art. Documents considered material to patentability will be separately identified by Information Disclosure Statement.

References

Ahrendt,S. A., Halachmi,S., Chow,J. T., Wu,L., Halachmi,N., Yang,S. C., Wehage,S., Jen,J. and Sidransky,D. (1999) Rapid p53 sequence analysis in primary lung cancer using an oligonucleotide probe array. Proc. Natl. Acad. Sci. USA, 96, 7382-7.
Baines, W., and Smith, G C., J. Theor. Biology, 135:303-307 (1988).
Cutler,D. J., Zwick,M. E., Carrasquillo,M. M., Yohn,C. T., Tobin,K. P., Kashuk,C., Mathews,D. J., Shah,N. A., Eichler,E. E., Warrington,J. A. et al. (2001) High-throughput variation detection and genotyping using microarrays. Genome Res., 11, 1913-25.
Drmanac, R., and Crkvenjakov, R., Yugoslav Patent Application 570 (1987).
Drmanac,S., Kita,D., Labat,I., Hauser,B., Schmidt,C., Burczak,J. D. and Drmanac,R. (1998) Accurate sequencing by hybridization for DNA diagnostics and individual genomics. Nat Biotechnol., 16, 54-8.
Drmanac,R., Drmanac,S., Baier,J., Chui,G., Coleman,D., Diaz,R., Gietzen,D., Hou,A., Jin,H., Ukrainczyk,T. et al. (2001) DNA sequencing by hybridization with arrays of samples or probes. Methods Mol. Biol., 170, 173-9.
Drmanac,R. and Drmanac,S. (2001) Sequencing by hybridization arrays. Methods Mol. Biol., 170, 39-51.
Drmanac,R., Drmanac,S., Chui,G., Diaz,R., Hou,A., Jin,H., Jin,P., Kwon,S., Lacy,S., Moeur,B. et al. (2002) Sequencing by hybridization (SBH): advantages, achievements, and opportunities. Adv. Biochem. Eng. Biotechnol., 77, 75-101.
Durbin, R., Eddy, S., Krogh, A., and Mitchison, G., Biological Sequence Analysis: Probabilistic Models of proteins and Nucleic Acids, Cambridge University Press, (1998).
Frieze,A. M., Preparata,E P. and Upfal E. (1999) Optimal reconstruction of a sequence from its probes. J. Comput. Biol., 6, 361-8.
Gunderson,K. L., Huang,X. C., Morris,M. S., Lipshutz,R. J., Lockhart,D. J. and Chee,M. S. (1998) Mutation detection by ligation to complete n-mer DNA arrays. Genome Res., 8, 1142-53.
Guo,Z., Gatterman,M. S., Hood,L., Hansen,J. A. and Petersdorf,E. W. (2002) Oligonucleotide Arrays for High-Throughput SNPs Detection in the MHC Class I Genes: HLA-B as a Model System. Genome Res., 12, 447-457.
Head,S. R., Rogers,Y. H., Parikh,K., Lan,G., Anderson,S., Goelet,P. and Boyce-Jacino,M. T. (1997) Nested genetic bit analysis (N-GBA) for mutation detection in the p53 tumor suppressor gene. Nucleic Acids Res., 25, 5065-71.
Head,S. R., Goelet,P., Karn,J. and Boyce-Jacino,M. (2001), U.S. Pat. No. 6,322,968.
Head,S. R., Goelet,P., Karn,J. and Boyce-Jacino,M. (2002) U.S. Pat. No. 6,337,188.
Khrapko, K. R., Lysov, Y P., Khorlyn, A. A., Shick, V V., Florentiev, V. L., and Mirzabekov, A. D., FEBS Letters, 256:118-122 (1989).
Kimura, M., Journal ofMolecular Evolution, 16:111-120 (1980).
Lebed,J. B., Chechetkin,V. R., Turygin,A. Y., Shick,V. V. and Mirzabekov,A. D. (2001) Comparison of complex DNA mixtures with generic oligonucleotide microchips. J. Biomol. Struct. Dyn., 18, 813-23.
Kozal,M. J., Shah,N., Shen,N., Yang,R., Fucini,R., Merigan,T. C., Richman,D. D., Morris,D., Hubbell,E., Chee,M. et al. (1996) Extensive polymorphisms observed in HIV-1 clade B protease gene using high-density oligonucleotide arrays. Nat Med., 2, 753-9.
Liu,Y., Hansen,E., Penney,R., Gelfand,C. A. and Boyce-Jacino,M. T. (2001) A Universal Assay for DNA Sequence Analysis and SNP Genotyping. Poster presented on the 13th International Conference on Genome Sequencing & Analysis, San Diego, Calif., USA.
Lysov, Y., Floretiev, V., Khorlyn, A., Khrapko, K., Shick, V, and Mirzabekov, A., Dokl, Acad. Sci., USSR, 303:1508-1511 (1988).
Macevices, S. C., International Patent Application PS US89 04741 (1989).
Pe'er,I. and Shamir,R. (2000). Spectrum alignment: efficient resequencing by hybridization. Proc. Int. Conf. Intell. Syst. Mol. Biol., 8, 260-8.
Pe'er, I., Arbili, N. and Shamir, R. (2002) A computational method for resequencing long DNA targets by universal oligonucleotide arrays. Proc. Natl. Acad. Sci. USA, 99, 15492-6.
Pe'er,I., Arbili,N., Liu,Y, Enck,C., Gelfand,C., and Shamir,R. (2003). Advanced Computational Techniques for Resequencing DNA with Polymerase Signaling Assay Arrays. Nucleic Acids Research 31(19):5667-75
Pevzner,P. A. (1989) 1-Tuple DNA sequencing: computer analysis. J. Biomol. Struct. Dyn., 7, 63-73.
Pevzner, P. A., and Lipshutz, R. J., Towards DNA Sequencing Chips. Mathematical Foundations of Computer Science, LNCS 841:143-158 (1994).
Pevzner, P. A., Lysov, Y P., Khrapko, K. R., Belyavsky, A. V., Florentiev, V. L., and Mirzabekov, A. D., J. Biomol. Struct. Dyn. 7:63-73 (1989).
Preparata, E, Frieze, A., and Upfal, E., Journal of Computational Biology 6(3-4):361-368 (1999).
Preparata,F. P. and Upfal,E. (2000) Sequencing-by-hybridization at the information-theory bound: an optimal algorithm. J. Comput. Biol., 7, 621-30.
Southern, E. M., Maskos, U., and Elder, J. K., Genomics 13:1008-1017 (1992).
Southern E., UK patent Application GB 8,810,400 (1988).
Southern, E. M., Trends in Genetics 12:110-115 (1996).
Tillib,S. V. and Mirzabekov,A. D. (2001) Advances in the analysis of DNA sequence variations using oligonucleotide microchip technology. Curr Opin Biotechnol., 12, 53-8.
Yan,H., Kinzler,K. W. and Vogelstein,B. (2000) Genetic Testing—Present and Future. Science, 289, 1890-1892.
The International SNP Map Working Group (2001) A map of human genome sequence variation containing 1.42 million single nucleotide polymorphisms.
Nature. 409, 928-33.
National Institute of Health (2002) Large scale genotyping for the haplotype map of the human genome. Request for application HG-02-005.
U.S. patent application Ser. No. 09/643,407

1. A method for obtaining a candidate nucleotide sequence S, the candidate nucleotide sequence S being indicative of a sequence of a target polynucleotide molecule \hat{T}, T producing a hybridization signal I(\vec{x}) upon incubating T with a polynucleotide \vec{x} for each polynucleotide \vec{x} in a set E of polynucleotides, the method comprising the steps of:
(a) for each polynucleotide \vec{x} in the set E of polynucleotides, obtaining a probability P₀(\vec{x}) of the hybridization signal I(\vec{x}) when the sequence \vec{x} is not complementary to a subsequence of T and a probability Pₗ(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
(ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;
(eb) selecting one or more candidate nucleotide sequences having an essentially maximal score; and
(ec) determining a revised candidate sequence S′ indicative of the sequence of the target polynucleotide molecule T by substituting the sequence of the low confidence region in the candidate sequence S with the candidate sequence selected in step (eb).

2. The method according to claim 1 further comprising repeating step e iteratively to the revised candidate sequence S′ determined in the previous iteration.

3. The method according to claim 1, wherein the polynucleotides \vec{x} in the set E are immobilized on a surface.

4. The method according to claim 1 wherein the set E is a set of k-mers.

5. The method according to claim 4 wherein E is the set of all k-mers formed from nucleotides from a predetermined set of nucleotides..

6. The method of claim 5 wherein the predetermined set of nucleotides is selected from the group consisting of
(a) adenine, guanine, cytosine, and thymine; and
(b) adenine, guanine, cytosine, uracil.

7. The method according to claim 1, wherein the score of a candidate nucleotide sequence \hat{T} is based upon L^{e}(\hat{T}) where
$${L}^{e} ( \hat{T} ) = {\prod }_{\vec{x} ∈ A}^{} {P}_{\hat{T} ( \overline{x} )} ( \vec{x} ) ,$$
wherein \vec{T}(\vec{x})=0 if the sequence of \vec{x} is not complementary to a subsequence of \hat{T} and \hat{T}(\vec{x})=1 if the sequence of \vec{x} is complementary to a subsequence of \hat{T}.

8. The method according to claim 1, wherein the score of a candidate sequence \hat{T} is based upon \tilde{L}^{e}(\hat{T}) where
$$log {\tilde{L}}^{e} ( \hat{T} ) = {\sum }_{i = 0}^{m} ω ( {e}_{i} ) ,$$
wherein \hat{T} contains polynucleotides eₒ, . . . eₘ and
$$ω ( {e}_{i} ) = log \frac{{P}_{1} ( {e}_{i} )}{{P}_{0} ( {e}_{i} )} .$$

9. The method according to claim 1, wherein the reference sequence is a hidden Markov model.

10. The method according to claim 9, wherein the score of a candidate sequence \hat{T} is based upon D^{u}(\hat{T}) where
$${D}^{u} ( \hat{T} ) = {\prod }_{j = 1}^{l} {M}^{( j )} [ {t}_{j} , {h}_{j} ] ,$$
wherein M^{(j)}[tⱼ, hⱼ] is a probability of a nucleotide tⱼ in position j of T being replaced with nucleotide hⱼ in position j of H.

11. The method according to claim 1 for use in a task selected from the group comprising:
(a) Detecting or genotyping;
(b) Detecting local mutations in the sequence;
(c) detecting single nucleotide polymorphisms, insertions or deletions;
(d) Detecting or genotyping of genetic syndroms or disorders.
(e) Detecting or genotyping somatic mutations.
(f) Sequencing a polynucleotide having a function that is related to a function of the reference polynucleotide.
(g) Sequencing a polynucleotide which is orthologous to a reference polynucleotide in another species;
(h) Sequencing double stranded DNA; and
(i) Detecting a heterozygote. .

12. The method according to claim 1, wherein polypeptides are sequenced instead of polynucleotides.

13. The method according to claim 1 wherein the probabilities P₀( \vec{x} ) and P₁( \vec{x} ) are determined by a probe-training method.

14. The method according to claim 13 wherein P₁(\vec{x}) is the p-value for a signal s(x) to be drawn from a normal distribution with mean μ₁(x) and standard deviation σ₁(x) wherein μ₁(x) is the mean signal of a matched probe and σ₁(x) is the standard deviation of the signal of a matched probe, wherein P₀( \vec{x} ) is the p-value for a signal s(x) to be drawn from a normal distribution with mean μ₀(x) and standard deviation σ₀(x) wherein μ₀(x) is a mean signal of an unmatched probe and σ₀(x) is the standard deviation of the signal of an unmatched probe.

15. The method according to claim 1 wherein the probabilities P₀( \vec{x} )and P₁(\vec{x}) are determined by a probe independent training method.

16. The method according to claim 15 comprising generating N candidate targets, and averaging the matched/unmatched signal of each probe.

17. The method according to claim 16 wherein averaging the matched/ unmatched status of the probe \vec{x} comprises estimating the probability of a perfect match based on the N candidate targets as the fraction of probes attaining a predetermined signal among perfectly matched probes and setting:
$$\begin{matrix}{P}_{1} ( x ) = \frac{{\sum }_{random target t}^{} ( {N}^{<} ( t , s ( x ) ) + 0.5 {N}^{=} ( t , s ( x ) ) )}{{\sum }_{random target t}^{} {N}^{<} ( t , ∞ )} \\ {P}_{1} ( x ) = \frac{ \begin{matrix}0.5 + \# Experiments with perfect \\ match for x and signal < s ( x )\end{matrix}}{1 + \# Experiments with perfect match for x}\end{matrix} \qquad ( Eq . 1 )$$
Please write out P₀(x) explicitly
where N^{<}(t,s) denotes the number of experiments perfectly matching x displaying a signal below s, and N⁼(t,s) denotes the number of experiments perfectly matching \vec{x} displaying a signal equal to s.

18. The method according to claim 1 wherein \vec{x} is a pool of nucleotides.

19. The method according to claim 1 wherein a low confidence interval is an interval having an average nucleotide score below a predetermined threshold.

20. A program storage device readable by machine, tangibly embodying a program of instruction executable by the machine to perform method steps for obtaining a candidate nucleotide sequence S, the candidate nucleotide sequence S being indicative of a sequence of a target polynucleotide molecule T, T producing a hybridization signal I(\vec{x}) upon incubating T with a polynucleotide \vec{x} for each polynucleotide \vec{x} in a set E of polynucleotides, the method comprising the steps of:
(a) for each polynucleotide \vec{x} in the set E of polynucleotides, obtaining a probability P₀( \vec{x} ) of the hybridization signal I( \vec{x} ) when the sequence \vec{x} is not complementary to a subsequence of T and a probability P₁(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
(ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;
(eb) selecting one or more candidate nucleotide sequences having an essentially maximal score; and
(ec) determining a revised candidate sequence S′ indicative of the sequence of the target polynucleotide molecule T by substituting the sequence of the low confidence region in the candidate sequence S with the candidate sequence selected in step (eb)..

21. A computer program product comprising a computer useable medium having computer readable program code embodied therein for obtaining a candidate nucleotide sequence S, the candidate nucleotide sequence S being indicative of a sequence of a target polynucleotide molecule T, T producing a hybridization signal I( \vec{x} ) upon incubating T with a polynucleotide \vec{x} for each polynucleotide \vec{x} in a set E of polynucleotides, thecomputer program product comprising:
(a) for each polynucleotide \vec{x} in the set E of polynucleotides, obtaining a probability P₀(\vec{x}) of the hybridization signal I(\vec{x}) when the sequence \vec{x} is not complementary to a subsequence of T and a probability Pₗ(\vec{x}) of the hybridization signal when the sequence \vec{x} is complementary to a subsequence of T; so as to obtain a probabilistic spectrum (PS) of T;
(b) assigning a score to each of a plurality of candidate nucleotide sequences, the score being based upon the probabilistic spectrum and upon a reference nucleotide sequence H;
(c) selecting one or more candidate nucleotide sequences having an essentially maximal score;
(d) detecting one or more low confidence intervals and one or more reliable intervals in the selected candidate nucleotide sequence; and
(e) For each of the one or more low confidence intervals detected in the selected candidate nucleotide sequence:
(ea) assigning a score to each of a plurality of candidate nucleotide sequences of the low confidence region, the score being based upon a probabilistic spectrum obtained by filtering from the PS signals the signals present in the reliable regions; and upon an interval of the reference nucleotide sequence H homologous with the low confidence interval;;
(eb) selecting one or more candidate nucleotide sequences having an essentially maximal score; and
(ec) determining a revised candidate sequence S′ indicative of the sequence of the target polynucleotide molecule T by substituting the sequence of the low confidence region in the candidate sequence S with the candidate sequence selected in step (eb).


SEQUENCE LISTING (42 sequences)

SEQ ID NO: 1 - DNA, 100 bp, Homo sapiens
attgacaggttcatgcaggctgtgacaggatggaagactggctgctccctgatgggagccagtgtggacagcaccctggctttcaacacctacgtccact

SEQ ID NO: 2 - DNA, 100 bp, Homo sapiens; identical to SEQ ID NO: 1

SEQ ID NO: 3 - DNA, 100 bp, Homo sapiens; identical to SEQ ID NO: 1

SEQ ID NO: 4 - DNA, 100 bp, Homo sapiens; identical to SEQ ID NO: 1

SEQ ID NO: 5 - DNA, 100 bp, Homo sapiens; identical to SEQ ID NO: 1

SEQ ID NO: 6 - DNA, 100 bp, Homo sapiens; identical to SEQ ID NO: 1

SEQ ID NO: 7 - DNA, 98 bp, Homo sapiens
ctagaagaggacatctccaagtttgcagagaaagacaatatagttcttggagaaggtggaatcacactgagtggaggtcaacgagcaagaatttcttt

SEQ ID NO: 8 - DNA, 110 bp, Homo sapiens
ccaagtttgcagagaaagacaatatagttcttggagaaggtggaatcacactgagtggaggtcaacgagcaagaatttctttagcaagagcagtatacaaagatgctgat

SEQ ID NO: 9 - DNA, 110 bp, Homo sapiens; identical to SEQ ID NO: 8

SEQ ID NO: 10 - DNA, 108 bp, Homo sapiens
tcttggagaaggtggaatcacactgagtggaggtcaacgagcaagaatttctttagcaagagcagtatacaaagatgctgatttgtatttattagactctccttttgg

SEQ ID NO: 11 - DNA, 25 bp, Homo sapiens
atatagttcttggagaaggtggaat

SEQ ID NO: 12 - DNA, 25 bp, Homo sapiens
atatagttcttggagaaggtggaat

SEQ ID NO: 13 - DNA, 24 bp, Homo sapiens
ttctttagcaagagcagtatacaa

SEQ ID NO: 14 - DNA, 98 bp, Homo sapiens; identical to SEQ ID NO: 7

SEQ ID NO: 15 - DNA, 110 bp, Homo sapiens; identical to SEQ ID NO: 8

SEQ ID NO: 16 - DNA, 110 bp, Homo sapiens; identical to SEQ ID NO: 8

SEQ ID NO: 17 - DNA, 108 bp, Homo sapiens; identical to SEQ ID NO: 10

SEQ ID NO: 18 - DNA, 23 bp, Homo sapiens
gagtggaggtcaacgagcaagaa

SEQ ID NO: 19 - DNA, 23 bp, Homo sapiens
gagtggaggtcaacgagcaagaa

SEQ ID NO: 20 - DNA, 24 bp, Homo sapiens
ttctttagcaagagcagtatacaa

SEQ ID NO: 21 - DNA, 110 bp, Homo sapiens; identical to SEQ ID NO: 8

SEQ ID NO: 22 - DNA, 110 bp, Homo sapiens; identical to SEQ ID NO: 8

SEQ ID NO: 23 - DNA, 90 bp, Homo sapiens
aatcacactgagtggaggtcaacgagcaagaatttctttagcaagagcagtatacaaagatgctgatttgtatttattagactctccttt

SEQ ID NO: 24 - DNA, 90 bp, Homo sapiens; identical to SEQ ID NO: 23

SEQ ID NO: 25 - DNA, 99 bp, Homo sapiens
gaaagacaatatagttcttggagaaggtggaatcacactgagtggaggtcaacgagcaagaatttctttagcaagagcagtatacaaagatgctgattt

SEQ ID NO: 26 - DNA, 98 bp, Homo sapiens; identical to SEQ ID NO: 7

SEQ ID NO: 27 - DNA, 108 bp, Homo sapiens; identical to SEQ ID NO: 10

SEQ ID NO: 28 - DNA, 98 bp, Homo sapiens; identical to SEQ ID NO: 7

SEQ ID NO: 29 - DNA, 98 bp, Homo sapiens; identical to SEQ ID NO: 7

SEQ ID NO: 30 - DNA, 98 bp, Homo sapiens; identical to SEQ ID NO: 7

SEQ ID NO: 31 - DNA, 110 bp, Homo sapiens; identical to SEQ ID NO: 8

SEQ ID NO: 32 - DNA, 110 bp, Homo sapiens; identical to SEQ ID NO: 8

SEQ ID NO: 33 - DNA, 90 bp, Homo sapiens; identical to SEQ ID NO: 23

SEQ ID NO: 34 - DNA, 90 bp, Homo sapiens; identical to SEQ ID NO: 23

SEQ ID NO: 35 - DNA, 108 bp, Homo sapiens; identical to SEQ ID NO: 10

SEQ ID NO: 36 - DNA, 108 bp, Homo sapiens; identical to SEQ ID NO: 10

SEQ ID NO: 37 - DNA, 35 bp, Homo sapiens
tgagtgagggctaagtttgatgcttactgtcccac

SEQ ID NO: 38 - DNA, 35 bp, Homo sapiens; identical to SEQ ID NO: 37

SEQ ID NO: 39 - DNA, 109 bp, Homo sapiens
atttagagtagtgggcaggttgaaaggatgtggacttcagaggtgagtgagggctaagtttgatgcttactgtcccacttataagctctatgtattcagccttgtttac

SEQ ID NO: 40 - DNA, 109 bp, Homo sapiens; identical to SEQ ID NO: 39

SEQ ID NO: 41 - DNA, 25 bp, Homo sapiens
atatagttcttggagaaggtggaat

SEQ ID NO: 42 - DNA, 25 bp, Homo sapiens
atatagttcttggagaaggtggaat